(19) United States
(12) Patent Application Publication
(10) Pub. No.: US 2024/0082071 A1
(43) Pub. Date: Mar. 14, 2024

(54) MACHINE FOR MAKING ABSORBENT SANITARY ARTICLES

(71) Applicant: GDM S.P.A., Bologna (IT)

(72) Inventors: Andrea FRIGENI, Bologna (IT); Matteo PIANTONI, Bologna (IT); Alessandro SACCOMANI, Bologna (IT); Alessandro ZAVALLONI, Bologna (IT)

(21) Appl. No.: 18/364,115

(22) Filed: Aug. 2, 2023

(30) Foreign Application Priority Data
Aug. 4, 2022 (IT) 102022000016608

(51) Int. Cl. A61F 13/15

(52) U.S. Cl. CPC A61F 13/15723; A61F 13/15772

(57) ABSTRACT

A machine for making absorbent sanitary articles, includes at least one cutting unit configured to divide a continuous web into segments intended for manufacturing an absorbent sanitary article;
the anvil member see includes a drum, rotating about a respective axis of rotation, for retaining the continuous web and the segments obtained therefrom;
the cutting member includes a first cutting roller and a second cutting roller, both acting in conjunction with the drum of the anvil member and each rotating about a respective axis of rotation.

### BACKGROUND OF THE INVENTION

This application claims priority to Italian Application No. 102022000016608 filed Aug. 4, 2022, which application is incorporated by reference herein.

This invention relates to a machine for making absorbent sanitary articles.

In particular, a machine for making absorbent sanitary articles comprises at least one cutting unit for cutting a continuous web into segments.

By way of example and without limiting the invention, the continuous web may be in the form of a web of non-woven fabric or a web comprising a polymeric material (for example, polyethylene or polypropylene) or a web comprising a natural or seminatural polymeric material (for example, cellulose and derivatives). Typically, the cutting unit comprises a cutting member and an anvil member acting in conjunction with each other.

The anvil member supports and feeds the continuous web which is being divided into segments by the cutting member.

Generally speaking, the cutting member is of a rotary type and comprises one or more cutting edges which impact the anvil member so that the continuous web supported by the anvil member is divided into segments.

The anvil member may also be of a rotary type and, in use, has a constant rotation speed.

In order to make a discrete segment of length equal to the size of the absorbent article to be made, the cutting member must make two suitably spaced, consecutive cuts and, therefore, during cutting, the cutting edge must move at the same rotation speed as the anvil member.

After making the cut, the law of motion of the cutting member may vary, meaning that it may accelerate and then decelerate when the cutting edge again needs to move at the same speed as the anvil member, or vice versa.

Nowadays, the cutting member is driven by an electric actuator and an electronic cam controls the speed of the cutting member according to a law of motion corresponding to a respective size of the absorbent article.

The electronic cam allows applying two or more laws of motion to the cutting member so that a single cutting unit can be used to make segments corresponding to two or more sizes of absorbent articles.

This has undoubted advantages in terms of reducing machine down times for size changeover operations.

Flexibility is not the only requirement to be met by current machines, which must also be able to operate at high production speeds.

This affects the choice of actuator for the cutting member which, because it has to follow the laws of motion applied by the electronic cam to range from a small size, denoted “S”, to a large size, denoted “L”, must necessarily be high-powered, thus involving high costs and a significant installation footprint.

It must therefore be borne in mind that the performance required of the machine in terms of production speed clashes with the need for flexibility.

Another solution adopted to meet the need for flexibility without having to reduce production speed is to provide the machine with two cutting units, each having a respective anvil member and cutting member, so as to be able to activate one or the other, depending on the size of the absorbent article to be made.

This solution not only has a higher cost than that with the single cutting unit but also involves machine down time when changing over from one cutting unit to the other.

Moreover, providing the machine with a plurality of cutting units, each having a respective anvil member and cutting member, inevitably increases the cost of constructional parts, including redundant parts, and of the drives needed for them.

### SUMMARY OF THE INVENTION

The Applicant has developed a machine for making absorbent sanitary articles, comprising at least one cutting unit configured to divide a continuous web into segments intended for manufacturing an absorbent sanitary article.

The cutting unit comprises an anvil member and a cutting member acting in conjunction with the anvil member.

The anvil member comprises a drum, rotating about a respective axis of rotation, for retaining the continuous web and the segments obtained therefrom.

The cutting unit comprises a control and drive unit configured to control the movement of the cutting member at least as a function of the rotation speed of the drum of the anvil member.

The cutting member comprises a first cutting roller and a second cutting roller, distinct from each other, both acting in conjunction with the drum of the anvil member and each rotating about a respective axis of rotation.

Actuating means are configured to set the first cutting roller in rotation about its axis of rotation via respective motion transmission means and to set the second cutting roller in rotation about its axis of rotation via respective motion transmission means.

The control and drive unit is configured to selectively command the actuation of the first cutting roller and the second cutting roller via the actuating means as a function of a first size range and a second size range of absorbent sanitary articles respectively, and to apply to the first cutting roller a law of motion corresponding to a respective size belonging to the first size range and to apply to the second cutting roller a law of motion corresponding to a respective size belonging to the second size range.

Each law of motion has respective accelerations and decelerations.

Advantageously, the possibility of dividing the laws of motion of the cutting member between two distinct cutting rollers allows meeting the need for flexibility without negatively impacting the production speed and thus improving the machine efficiency compared to the prior art solutions.

Another advantage is due to the limited size of the transmission systems, which in turn translates as lower costs, lower consumption and smaller footprint compared to the prior art solutions.

Advantageously, moreover, changing over to a different size does not require any additional operation.

Advantageously, each cutting rollers corresponds to specific laws of motion, thereby maximizing performance in terms of the speed of each roller.

Advantageously, once a size range processable by a cutting unit of known type has been defined, partializing this range into two or more ranges allows increasing production speeds without increasing the power of the drive systems.

Preferably, the control and drive unit is configured to be programmed from the outside and to receive instructions relating to the laws of motion of the actuating means.

Advantageously, the settings of the control and drive unit are based on the sizes of the absorbent article to be processed.

Preferably, the cutting unit comprises first actuating means for actuating the first cutting roller and second actuating means for actuating the second cutting roller.

Advantageously, by dedicating a respective actuator to each cutting roller maximizes performance in terms of achievable production speeds and minimizes size changeover times.

Alternatively, the actuating means are configured to move either the first cutting roller or the second cutting roller. Advantageously, it is possible to use a single actuator, to be connected to the first cutting roller or to the second cutting roller based on the sizes to be processed.

Preferably, the actuating means comprise first actuating means for actuating the first cutting roller and second actuating means for actuating the second cutting roller.

This invention provides a method for making absorbent sanitary articles, comprising a step of cutting to divide a continuous web into segments intended for manufacturing an absorbent sanitary article.

The step of cutting comprises selectively commanding the rotation of one between a first cutting roller and a second cutting roller, both acting in conjunction with the same anvil member, as a function of a respective first size range and of a second size range of absorbent sanitary articles, and applying the rotation speed to one between the first cutting roller or the second cutting roller.

In the step of cutting, while one between the first cutting roller and the second cutting roller rotates about a respective axis of rotation, the other remains in a paused condition.

The step of cutting comprises applying a law of motion to the rotation of the first cutting roller or of the second cutting roller corresponding to a respective size belonging to the first size range or to the second size range, each law of motion having respective accelerations and decelerations.

Further features and advantages of the invention are more apparent in the exemplary, hence non-limiting description which follows of a preferred but non-exclusive embodiment of a machine for making absorbent sanitary articles and of the related method.

### BRIEF DESCRIPTION OF THE DRAWINGS

The description is set out below with reference to the accompanying drawings which are provided solely for purposes of illustration without restricting the scope of the invention and in which:

FIG. 1 is a schematic front view illustrating a machine for making absorbent sanitary articles according to this invention;

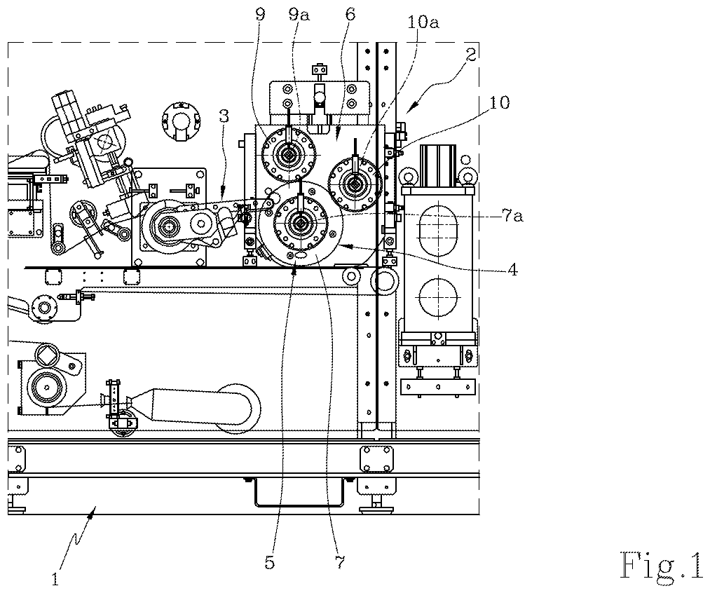

FIG. 2 is a schematic perspective view of a cutting unit of the machine of FIG. 1.

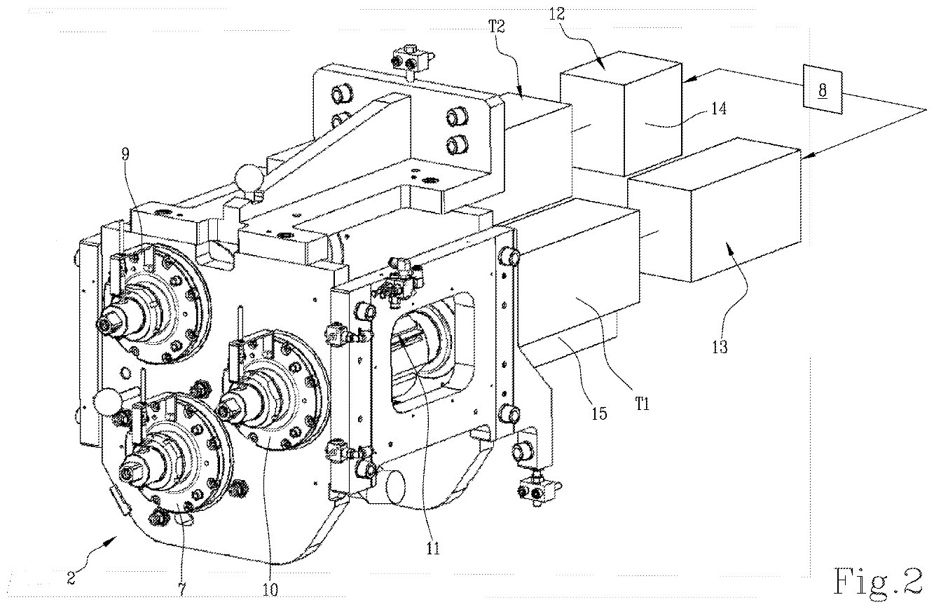

### DESCRIPTION OF THE PREFERRED EMBODIMENTS

The reference numeral 1 denotes a machine for making absorbent sanitary articles according to this invention.

The machine 1 comprises at least one cutting unit 2 configured to divide a continuous web 3 into segments 4 intended for manufacturing an absorbent sanitary article.

The cutting unit 2 comprises an anvil member 5 and a cutting member 6 acting in conjunction with the anvil member 5.

The anvil member 5 comprises a drum 7, rotating about a respective axis of rotation 7a, for retaining the continuous web 3 and the segments 4 obtained therefrom.

The cutting unit 2 comprises a control and drive unit 8 configured to control the movement of the cutting member 6 at least as a function of the rotation speed of the drum 7 of the anvil member 5.

The cutting member 2 comprises a first cutting roller 9 and a second cutting roller 10, both acting in conjunction with the drum 7 of the anvil member 5 and each rotating about a respective axis of rotation 9a, 10a.

The first cutting roller 9 and the second cutting roller 10 each comprise respective cutting members 11 driven in rotation about respective axes of rotation 9a, 10a.

The control and drive unit 8 is configured to control the rotation speed of the first cutting roller 9 or, alternatively, the rotation speed of the second cutting roller 10 in relation to a respective size of an absorbent article to be made.

The control and drive unit 8 is configured to control the rotation speed of the first roller 9 as a function of a first size range or, alternatively, to control the rotation speed of the second roller 10 as a function of a second size range.

Advantageously, the control and drive unit 8 is configured to control the rotation speed of the first cutting roller 9 or, alternatively, the rotation speed of the second cutting roller 10 according to the size to be processed.

Thus, the first cutting roller 9 and the second roller 10 can rotate at the maximum speeds permitted by the respective actuators since they are controlled by respective laws of motion corresponding to the sizes associated with the first roller 9 and the second roller 10.

The control and drive unit 8 is configured to control the rotation speed of the first roller 9 or of the second roller 10 according to a law of motion corresponding to a respective size belonging to the first size range or to the second size range; each law of motion has respective accelerations and decelerations.

The control and drive unit 8 is configured to receive instructions relating to the laws of motion to be applied to the first roller 9 and to receive instructions relating to the laws of motion to be applied to the second roller 10.

The control and drive unit 8 is configured to be programmed from the outside and to receive instructions relating to the laws of motion of the actuating means 13, 14.

For example, the instructions may be entered using a control panel installed in the machine 1.

In a first variant embodiment, the machine 1 comprises first actuating means 14 for actuating the first cutting roller 9 and second actuating means 13 for actuating the second cutting roller 10.

Preferably, the actuators, 13, 14 are electric actuators.

In a second variant embodiment, the machine 1 comprises actuating means 14, 13 configured to move the first cutting roller 9 or, alternatively, the second cutting roller 10.

In this embodiment, if the roller being moved is the first cutting roller 9 to make the sizes corresponding to the first size range, the actuating means 14 are associated with the first cutting roller 9.

If the roller being moved is the second cutting roller 10 to make the sizes corresponding to the second size range, the actuating means 14 are associated with the second cutting roller 10.

The actuating means 13, 14 are configured to set the first cutting roller 9 in rotation about its axis of rotation 9a via respective motion transmission means T1 and to set the second cutting roller 10 in rotation about its axis of rotation 10a via respective motion transmission means T2.

The control and drive unit 8 is configured to selectively command the actuation of the first cutting roller 9 and the second cutting roller 10 via the actuating means 13, 14 as a function of a first size range and a second size range of absorbent sanitary articles respectively, and to apply to the first cutting roller 9 a law of motion corresponding to a respective size belonging to the first size range and to impart to the second cutting roller 10 a law of motion corresponding to a respective size belonging to the second size range; each law of motion has respective accelerations and decelerations.

The machine 1 comprises actuating means 15 for actuating the drum 7 for retaining the continuous web 3 and the segments 4 obtained therefrom.

Preferably, the actuating means mentioned in this disclosure are electric actuators.

This invention also relates to a method for making absorbent sanitary articles, comprising a step of cutting configured to divide a continuous web 3 into segments 4 intended for manufacturing an absorbent sanitary article.

The step of cutting comprises controlling the rotation speed of a first cutting roller 9 or of a second cutting roller 10, both acting in conjunction with the same anvil member 5, as a function of a respective first size range and of a second size range of absorbent sanitary articles.

The step of cutting comprises selectively commanding the rotation of one between a first cutting roller 9 and a second cutting roller 10, both acting in conjunction with the same anvil member 5, as a function of a respective first size range and of a second size range of absorbent sanitary articles, and applying the rotation speed to one between the first cutting roller 9 or the second cutting roller 10.

In the step of cutting, while one between the first cutting roller 9 and the second cutting roller 10 rotates about a respective axis of rotation 9a, 10a, the other remains in a paused condition.

The step of cutting comprises applying a law of motion to the rotation of the first cutting roller 9 or of the second cutting roller 10 corresponding to a respective size belonging to the first size range or to the second size range, each law of motion having respective accelerations and decelerations.

1. A machine for making absorbent sanitary articles,
comprising at least one cutting unit configured to divide a continuous web into segments intended for manufacturing an absorbent sanitary article;
the cutting unit comprises an anvil member and a cutting member acting in conjunction with the anvil member;
the anvil member, rotating about a respective axis of rotation, for retaining the continuous web and the segments obtained therefrom;
the cutting unit comprises a control and drive unit configured to control the movement of the cutting member at least as a function of the rotation speed of the drum of the anvil member;
characterized in that
the cutting member comprises a first cutting roller and a second cutting roller, distinct from each other, both acting in conjunction with the drum of the anvil member and each rotating about a respective axis of rotation;
actuating means are configured to set the first cutting roller in rotation about its axis of rotation via respective motion transmission means and to set the second cutting roller in rotation about its axis of rotation via respective motion transmission means;
the control and drive unit is configured to selectively command the actuation of the first cutting roller and the second cutting roller via the actuating means as a function of a first size range and a second size range of absorbent sanitary articles respectively, and to impart to the first cutting roller a law of motion corresponding to a respective size belonging to the first size range and to apply to the second cutting roller a law of motion corresponding to a respective size belonging to the second size range; each law of motion has respective accelerations and decelerations.

2. The machine according to claim 1,
wherein the control and drive unit is configured to be programmed from the outside and to receive instructions relating to the laws of motion of the actuating means.

3. The machine according to claim 1, wherein the actuating means are configured to move the first cutting roller or, alternatively, the second cutting roller.

4. The machine according to claim 1, wherein the actuating means comprise first actuating means for actuating the first cutting roller and second actuating means for actuating the second cutting roller.

5. A method for making absorbent sanitary articles,
comprising a step of cutting to divide a continuous web into segments intended for manufacturing an absorbent sanitary article;
the step of cutting comprises selectively commanding the rotation of one between a first cutting roller and a second cutting roller, both acting in conjunction with the same anvil member, as a function of a respective first size range and of a second size range of absorbent sanitary articles, and applying the rotation speed to one between the first cutting roller or the second cutting roller;
in the step of cutting, while one between the first cutting roller and the second cutting roller rotates about a respective axis of rotation, the other remains in a paused condition;
the step of cutting comprises applying a law of motion to the rotation of the first cutting roller or of the second cutting roller corresponding to a respective size belonging to the first size range or to the second size range, each law of motion having respective accelerations and decelerations.
